SEQUENCE LISTING (27 sequences)

SEQ ID NO: 1 - RNA, 74 nt, Ganoderma lucidium
  source 1..74: mol_type = genomic RNA
aagcctataatttaaaggtagaataatttcttgataaggaatctgtagaagttcgattcttcttgggcttacca

SEQ ID NO: 2 - RNA, 33 nt, synthetic construct
  source 1..33: mol_type = other RNA
aagcctataatttaaaggtagaataatttcttg

SEQ ID NO: 3 - RNA, 41 nt, synthetic construct
  source 1..41: mol_type = other RNA
ataaggaatctgtagaagttcgattcttcttgggcttacca

SEQ ID NO: 4 - RNA, 38 nt, synthetic construct
  source 1..38: mol_type = other RNA
ataaggaatctgtagaagttcgattcttcttgggctta

SEQ ID NO: 5 - RNA, 22 nt, synthetic construct
  source 1..22: mol_type = other RNA
aagcctataatttaaaggtaga

SEQ ID NO: 6 - RNA, 19 nt, synthetic construct
  source 1..19: mol_type = other RNA
aagcctataatttaaaggt

SEQ ID NO: 7 - RNA, 22 nt, synthetic construct
  source 1..22: mol_type = other RNA
tcgattcttcttgggcttacca

SEQ ID NO: 8 - RNA, 19 nt, synthetic construct
  source 1..19: mol_type = other RNA
attcttcttgggcttacca

SEQ ID NO: 9 - RNA, 22 nt, synthetic construct
  source 1..22: mol_type = other RNA
agttcgattcttcttgggctta

SEQ ID NO: 10 - RNA, 19 nt, synthetic construct
  source 1..19: mol_type = other RNA
tcgattcttcttgggctta

SEQ ID NO: 11 - RNA, 33 nt, synthetic construct
  source 1..33: mol_type = other RNA
caagaaattattctacctttaaattataggctt

SEQ ID NO: 12 - RNA, 41 nt, synthetic construct
  source 1..41: mol_type = other RNA
tggtaagcccaagaagaatcgaacttctacagattccttat

SEQ ID NO: 13 - RNA, 38 nt, synthetic construct
  source 1..38: mol_type = other RNA
taagcccaagaagaatcgaacttctacagattccttat

SEQ ID NO: 14 - RNA, 22 nt, synthetic construct
  source 1..22: mol_type = other RNA
tctacctttaaattataggctt

SEQ ID NO: 15 - RNA, 19 nt, synthetic construct
  source 1..19: mol_type = other RNA
acctttaaattataggctt

SEQ ID NO: 16 - RNA, 22 nt, synthetic construct
  source 1..22: mol_type = other RNA
tggtaagcccaagaagaatcga

SEQ ID NO: 17 - RNA, 19 nt, synthetic construct
  source 1..19: mol_type = other RNA
tggtaagcccaagaagaat

SEQ ID NO: 18 - RNA, 22 nt, synthetic construct
  source 1..22: mol_type = other RNA
taagcccaagaagaatcgaact

SEQ ID NO: 19 - RNA, 19 nt, synthetic construct
  source 1..19: mol_type = other RNA
taagcccaagaagaatcga

SEQ ID NO: 20 - RNA, 41 nt, synthetic construct
  modified_base 19: mod_base = OTHER; note = u=m5u
  modified_base 20: mod_base = OTHER; note = n=p
  source 1..41: mol_type = other RNA
ataaggaatctgtagaagtncgattcttcttgggcttacca

SEQ ID NO: 21 - RNA, 41 nt, synthetic construct; identical to SEQ ID NO: 3
  modified_base 19: mod_base = OTHER; note = u=m5u
  source 1..41: mol_type = other RNA

SEQ ID NO: 22 - RNA, 41 nt, synthetic construct; identical to SEQ ID NO: 20
  modified_base 20: mod_base = OTHER; note = n=p
  source 1..41: mol_type = other RNA

SEQ ID NO: 23 - RNA, 22 nt, synthetic construct
  modified_base 1: mod_base = OTHER; note = n=p
  source 1..22: mol_type = other RNA
ncgattcttcttgggcttacca

SEQ ID NO: 24 - RNA, 41 nt, synthetic construct; identical to SEQ ID NO: 12
  source 1..41: mol_type = other RNA

SEQ ID NO: 25 - RNA, 41 nt, synthetic construct; identical to SEQ ID NO: 12
  source 1..41: mol_type = other RNA

SEQ ID NO: 26 - RNA, 41 nt, synthetic construct; identical to SEQ ID NO: 12
  source 1..41: mol_type = other RNA

SEQ ID NO: 27 - RNA, 22 nt, synthetic construct
  source 1..22: mol_type = other RNA
tggtaagcccaagaagaatcga

(19) United States
(12) Patent Application Publication
(10) Pub. No.: US 2025/0304965 A1
(43) Pub. Date: Oct. 2, 2025

(54) DOUBLE-STRANDED RNA MOLECULE AND PHARMACEUTICAL USE THEREOF

(71) Applicant: MACAU UNIVERSITY OF SCIENCE AND TECHNOLOGY, Macao (CN)

(72) Inventors: Zhihong Jiang, Macao (CN); Fei Ren, Macao (CN); Kaiyue Cao, Macao (CN); Ruize Gong, Macao (CN)

(73) Assignee: MACAU UNIVERSITY OF SCIENCE AND TECHNOLOGY, Macao (CN)

(21) Appl. No.: 18/863,988

(22) PCT Filed: May 11, 2023
(86) PCT No.: PCT/CN2023/093406

§ 371 Date: Nov. 7, 2024

(30) Foreign Application Priority Data
May 11, 2022 (CN) 202210514529.X

(51) Int. Cl. C12N 15/113

(52) U.S. Cl. CPC C12N 15/1135; C12N 2310/14; C12N 2310/335

(57) ABSTRACT

Provided are a double-stranded RNA molecule and pharmaceutical use thereof. The RNA is derived from Ganoderma lucidum (Curtis:Fr.) P. Karst., and has a prophylactic and therapeutic effect on tumors.

### CROSS-REFERENCE TO RELATED APPLICATIONS

This disclosure claims priority to Chinese Patent Application No. 202210514529.X, filed on May 11, 2022, which is incorporated herein by reference in its entirety.

### TECHNICAL FIELD

This disclosure relates to the field of biomedical technologies, and specifically, to a double-stranded RNA molecule and pharmaceutical use thereof.

### BACKGROUND

Cancers have become the commonest fatal diseases worldwide. The efficacy of small molecules such as alkaloids, terpenoids, and flavonoids in treatment of cancers has been proven. Some alkaloids have also been found to have effects on promoting inhibition of cancers by enhancing, for example, efficacy of anticancer medicants. However, most alkaloids are usually toxic to humans. In addition, it is usually believed that macromolecules such as DNA, RNA, and proteins are unstable and have poor activity in living humans. Therefore, it is not widely believed that the macromolecules are adequate in cancer treatment.

Currently, some studies have shown that small non-coding RNAs (small ncRNAs) such as microRNAs targeting different aspects of an RNA transcription or post-transcriptional process in almost all eukaryotes, exerting different regulatory effects. Mlotshwa, S. et al. (Cell research 2015, 25 (4), 521-4) have reported that exogenous plant microRNAs in food can be absorbed in digestive tracts of mammals and transferred through bloodstreams to various tissue cells in which the exogenous plant microRNAs can regulate expression of mammalian genes. Goodarzi, H. et al. (Cell 2015, 161 (4), 790-802) have disclosed that fragments derived from endogenous tRNAs can bind to RNA-binding proteins associated with pathogenesis and antagonize activity thereof, to inhibit stability of multiple oncogenic transcripts in breast cancer cells.

Ganoderma lucidum (Curtis: Fr.) P. Karst. is a species in the Ganodermataceae family (Ganodermataceae Donk). As a dominant species in the Yellow River basin, Ganoderma lucidum (Curtis: Fr.) P. Karst. is widely distributed throughout China and grows on oak trees, quercus trees, and other living broad-leaved trees or on stumps of dead trees. As early as the era of the Yellow Emperor between 2550 BC and 2140 BC, there had been records of Ganoderma lucidum (Curtis: Fr.) P. Karst. As important medicinal fungi, Ganoderma lucidum (Curtis: Fr.) P. Karst. contains compounds such as polysaccharides, triterpenoids, and steroids that have been widely studied and applied to development of medicants such as antibacterial, anticancer, anti-aging, anti-inflammatory, and immunomodulatory medicants.

However, there is still a need to extract efficacious molecules from various sources such as the medicinal fungus Ganoderma lucidum (Curtis: Fr.) P. Karst. beneficial to the human body to treat cancers.

### SUMMARY

A first purpose of this disclosure is to provide a double-stranded RNA molecule, consisting of an antisense strand and a sense strand hybridized thereto, wherein a nucleotide sequence of the antisense strand is denoted as any one of SEQ ID NOs: 2-10.

A second purpose of this disclosure is to provide a pharmaceutical composition, comprising the above double-stranded RNA molecule.

A third purpose of this disclosure is to provide use of the above double-stranded RNA molecule in preparation of a medicant for preventing or treating tumors.

The double-stranded RNA molecule provided in this disclosure can be used to prevent and treat tumors.

### BRIEF DESCRIPTION OF DRAWINGS

To describe the technical solutions in the specific embodiments of this disclosure or in the prior art more clearly, the following briefly describes the accompanying drawings required for describing the specific embodiments or the prior art. Apparently, the accompanying drawings in the following description show merely some embodiments of this disclosure, and those of ordinary skills in the art may still derive other drawings from these accompanying drawings without creative efforts.

FIG. 1 shows a gel electrophoresis spectrum of RNA molecules derived from Ganoderma lucidum (Curtis: Fr.) P. Karst., including a small RNA standard reference substance (denoted as a “Ladder”), a small RNA component, and a transfer RNA enrichment fragment, according to an exemplary embodiment.

FIG. 2 shows a bar chart (panel A) of read length distribution of transfer RNAs derived from Ganoderma lucidum (Curtis: Fr.) P. Karst. and a bar chart (panel B) of the numbers of reads of different types of transfer RNAs according to an exemplary embodiment.

FIG. 3A shows a spectrum of tRNA^{Ile(GAU)} separated in an ion-pair high-performance liquid chromatography method under 260 nm ultraviolet, a distribution diagram of multiple electric charges of tRNA^{Ile(GAU)} analyzed by ultra-performance liquid chromatography-mass spectrometry, and a spectrum of deconvolution of tRNA^{Ile(GAU)} according to an exemplary embodiment.

FIG. 3B shows a spectrum of tRNA^{Ile(GAU)} analyzed in the urea-denaturing polyacrylamide gel electrophoresis method, including a small RNA standard reference substance (denoted as “Ladder”), a microRNA standard reference substance (denoted as “microRNA Ladder”), a small RNA component (denoted as “small RNA”), and a tRNA^{Ile(GAU)} component according to an exemplary embodiment.

FIG. 4 shows a cleavage rule of oligonucleotide mass spectrometry applied to a method of characterizing purified tRNAs according to an exemplary embodiment.

FIG. 5 shows identification of characteristic fragments of tRNA^{Ile(GAU)} generated via RNA endonuclease T1 under 260 nm ultraviolet in the spectrum according to an exemplary embodiment.

FIG. 6 shows a curve graph of viability of A2780 cells, HCT-8 cells, and HepG2 cells treated with double-stranded RNA molecules GL1, GL2, GL4, and GL6 at different concentrations of 6.25 nM, 12.5 nM, 25 nM, 50 nM, 100 nM, and 200 nM that are derived from Ganoderma lucidum (Curtis: Fr.) P. Karst. in comparison with a control group and a liposome group according to an exemplary embodiment.

FIG. 7 shows a curve graph of viability of A2780 cells, HCT-8 cells, and HepG2 cells treated with double-stranded RNA molecules GL3, GL5, GL7, GL8, and GL9 at different concentrations that are derived from Ganoderma lucidum (Curtis: Fr.) P. Karst. in comparison with a control group and a liposome group according to an exemplary embodiment.

FIG. 8 shows a curve graph of viability of A2780 cells, HCT-8 cells, and HepG2 cells treated with double-stranded RNA molecules GL2, GL10, GL11, and GL12 at different concentrations of 6.25 nM, 12.5 nM, 25 nM, 50 nM, 100 nM, and 200 nM that are derived from Ganoderma lucidum (Curtis: Fr.) P. Karst. in comparison with a control group and a liposome group according to an exemplary embodiment.

### DETAILED DESCRIPTION

Reference is made in detail to embodiments of this disclosure, and one or more examples are described below. Each example is provided to illustrate rather than limit this disclosure. Actually, it is obvious for those skilled in the art to make various modifications and changes in this disclosure without departing from the scope or spirit of this disclosure. For example, features illustrated or described as a part of an embodiment can be used in another embodiment to create still another embodiment.

Unless otherwise specified, all terms (including technical and scientific terms) used to disclose this disclosure have the same meanings as commonly understood by those ordinary skills in the art to which this disclosure belongs. With reference to further guidance, the following definitions are used to better understand instructions in this disclosure. The terms used herein in the description of this disclosure are only for a purpose of describing specific embodiments, and are not intended to limit this disclosure.

A selection range of a term “and/or” used herein includes any one of two or more related listed items, and a combination of any one or all of the related listed items, and the combination of any one or all of the related listed items includes a combination of any two or more related listed items or all the related listed items. It should be noted that when at least three items are conjoined by “and/or”, it should be understood that, in this application, technical solutions undoubtedly include technical solutions logically conjoined by “and”, and also undoubtedly include technical solutions logically conjoined by “or”. For example, “A and/or B” includes three parallel solutions: A, B, and both A and B. For another example, technical solutions of “A, B, C and/or D” include any one of A, B, C or D (namely, a technical solution logically conjoined by “or”), and also include a combination of any one and all of A, B, C and D, that is, a combination of any two or three of A, B, C and D and a combination of four of A, B, C and D (namely, technical solutions logically conjoined by “or”).

Terms “comprise”, “include”, and “contain” used in this disclosure are synonymous, and are inclusive or open-ended, and do not exclude additional uncited members, elements, or method steps.

A value range in this disclosure that is defined by endpoints includes all values, fractions and the cited endpoints subsumed within the range.

A concentration value involved in this disclosure may fluctuate within a specific range. For example, the concentration value may fluctuate within a corresponding precision range. For example, the precision range of 2% allows fluctuation within a range of ±0.1%. A larger fluctuation is also allowed for a larger value or a value that does not require fine control. For example, a precision range of 100 mM can allow fluctuation within a range of ±1%, ±2%, ±5%, and so on. Molecular weight is allowed fluctuation of ±10%.

In this disclosure, descriptions such as “multiple” and “various” refer to two or more than two unless otherwise specified.

In this disclosure, technical features in open-ended descriptions include closed-ended technical solutions composed of listed features, and also include open-ended technical solutions including the listed features.

In this disclosure, “preferred”, “better”, “preferable” and “proper” are only intended to describe embodiments or examples with better effects, and should not be construed as a limitation on the protection scope of this disclosure. In this disclosure, “optionally”, “optional” and “option” mean “inessential”, that is, mean either of two parallel solutions of “with” or “without”. If there are multiple “options” in a technical solution, unless otherwise specified, if there is no contradiction or mutual constraint, each “option” is independent.

All documents mentioned in this disclosure are cited as references in this application, as if each document were individually cited as reference. Unless in conflict with the inventive objective and/or the technical solution of this application, the cited references involved in this disclosure are cited in their entireties and for all the objectives. When this disclosure involves the reference, definitions of a relevant technical characteristic, term, noun, phrase, and the like in the reference are also cited. When this disclosure involves the reference, examples and preferred methods of the cited relevant technical characteristic may also be incorporated into this application as a reference, provided that this disclosure can be achieved. It should be understood that when the cited content conflicts with the description in this application, this application shall prevail or be amended adaptively based on the description of this application.

This disclosure relates to a double-stranded RNA molecule, consisting of an antisense strand and a sense strand hybridized thereto, wherein a nucleotide sequence of the antisense strand is denoted as any one of SEQ ID NOs: 2-10.

In this disclosure, the term “hybridization” refers to formation of a duplex structure by two single-stranded nucleic acids through complementary base pairing. The hybridization can occur between completely complementary nucleic acid strands or between “basically complementary” nucleic acid strands that contain small mispairing regions. Conditions of allowing only hybridization of completely complementary nucleic acid strands are referred to as “strict hybridization conditions” or “sequence-specific hybridization conditions”. Stable duplexes of basically complementary sequences can be obtained under less strict hybridization conditions. Based on guidance provided in the field (referring to, for example, Sambrook et al., 2nd Edition 1989, Part 1-3, Molecular Cloning-A Laboratory Manual, Cold Spring Harbor Laboratory, Cold Spring Harbor, New York), technicians in the field of nucleic acid technologies can empirically consider multiple variables, including, for example, lengths of oligonucleotides and concentrations of base pairs, ionic strength, and incidence of mispaired base pairs to determine stability of duplexes.

The strict conditions are usually selected to be approximately 5° C. lower than a melting temperature (Tm) for a specific sequence under specified ionic strength and pH. Tm is a temperature at which 50% of the duplexes is dissociated (under the specified ionic strength and pH). Lowering the strictness of the hybridization conditions allows tolerance of sequence mispairing. A mispairing tolerance degree can be controlled by properly adjusting the hybridization conditions.

In some embodiments, sense chains corresponding to the antisense chains denoted as SEQ ID NOs: 2-10 are denoted as SEQ ID NOs: 11-19, respectively.

It should be noted that, in one aspect, useful antisense strands include functional variants or homologs of the sequences provided as SEQ ID NOs: 2-10. The functional variants or homologs have nucleotide sequences sharing greater than 80%, 81%, 82%, 83%, 84%, 85%, 86%, 87%, 88%, 89%, 90%, 91%, 92%, 93%, 94%, 95%, 96%, 97%, 98%, or 99% identity. Such RNA modifications are also considered and can be prepared based on standard techniques. The term “identity percentage” in the context of two or more nucleotide sequences indicates that two or more sequences or subsequences have identical nucleotides or have nucleotides a specific percent of which are identical, which is measured by one of the following sequence comparison algorithms or through visual inspection when in comparison and alignment for maximum correspondence. For example, the identity percentage is calculated relative to entire lengths of coding regions of sequences to be compared. For sequence comparison, one sequence is usually used as a reference sequence, and a tested sequence is compared with the sequence. When a sequence comparison algorithm is used, the tested sequence and the reference sequence are input into a computer, and if required, coordinates of the subsequences are specified, and parameters of a sequence algorithm program are specified. The sequence comparison algorithm then is used to calculate an identity percentage of the tested sequence relative to the reference sequence based on the specified program parameters. The identity percentage can be determined by using search algorithms such as BLAST and PSI-BLAST (Altschul et al., 1990, J Mol Biol 215:3, 403-410; Altschul et al., 1997, Nucleic Acids Res25: 17, 3389-402).

The RNA molecule in this disclosure and its functional variants or homologs are preferably extracted from or derived from fungi of the Ganoderma genus. In an embodiment, the RNA molecule is extracted from or derived from Ganoderma lucidum (Curtis: Fr.) P. Karst. In some embodiments, the functional variant or homolog is derived from a sequence denoted as the following SEQ ID NO: 1:

(tRNA^{Ile(GAU)}):
SEQ ID NO: 1
AAGCCUAUAAUUUAAAGGUAGAAUAAUUUCUUGAUAAGGAAUCUGUAGAA
GUUCGAUUCUUCUUGGGCUUACCA

More specifically, the functional variants or homologs are classified into the following two categories: The first category are 5′-tRFs, including 5′ end of a mature tRNA sequence and a fragment with a length of 2 to 35 nucleotides formed through cleavage of a D ring, a D-ring arm, an anticodon loop, or an anticodon ring arm; and the second category are 3′-tRFs, including 3′-CCA end of a mature tRNA sequence and a fragment with a length of 2 to 35 nucleotides formed through cleavage of a T ring, a T-ring arm, an anticodon loop, or an anticodon ring arm. For example, tRFs obtained from tRNA^{Ile(GAU)} includes 5′-tRFs “AAGCCUAUAAUUAUAAAGGUAGA” with a length of 22 nucleotides, and 3′-tRFs “UCGAUUCUUCUUGGGCUUACCA” with a length of 22 nucleotides.

In some embodiments, the double-stranded RNA molecule also includes 3′ overhang. Preferably, the double-stranded RNA molecule includes 3′ overhang with 2 nucleotides. Providing the 3′ overhang improves stability of the RNA molecule.

In some embodiments, the antisense strand and/or the sense strand of the double-stranded RNA molecule includes one, two or more modified nucleotides.

In some embodiments, the modified nucleotide comprises one, two or more of 4-acetylcytidine, 5-(carboxyhydroxymethyl)uridine, dihydrouridine, 2′-O-methylpseudouridine, β,D-galactose Q nucleoside, 2′-O-methylguanosine, inosinate, N⁶-isoprenyladenosine, 1-methyl adenosine, 1-methyl pseudouridine, 1-methylinosine, 2′2-dimethyladenosine, 2-methyladenosine, 2-methylguanosine, 5-methyluridine, 3-methylcytidine, 5-methylcytidine, N⁶-methyladenosine, 7-methylguanosine, 5-methylaminomethyluridine, 5-carboxymethylaminomethyluridine, 5-carboxy methylaminomethyl-2-thiouridine, β,D-mannose Q nucleoside, 5-methoxycarbonylmethyl-2-thiouridine, 5-(methoxycarbonyl)methyluridine, 5-methoxyuridine, 2-thiomethyl-N⁶-isoprenyl adenosine, N-[(9-β-D-ribofuranosyl-2-methylthiopurin-6-yl) carbamoyl]threonine, N-[(9-β-D-ribo furanosylpurin-6-yl)N-methylcarbamoyl]threonine, uridine 5-oxyacetic acid methyl ester, uridine-5-oxyacetic acid, wybutoxosine, pseudouridine, Q nucleoside, 2-thiocytidine, 5-methyl-2-thiouridine, 2-thiouridine, 4-thiouridine, 5-thiouridine, N-[(9-β-D-ribofuranosyl-6-yl) carbamoyl] threonine, 2′-O-methyl adenosine-5-methyluridine, 2′-O-methyladenosine, 2′-O-methylcytidine, wybutosine, 3-(3-amino-3-carboxy-propyl)uridine, N⁶-acetyladenosine, and 2-methylthio-N⁶-methyladenosine.

In some embodiments, a nucleotide sequence of the antisense strand of the double-stranded RNA molecule is denoted as any one of SEQ ID NOs: 20-23, and a nucleotide sequence of a corresponding sense strand is denoted as any one of SEQ ID NOs: 24-27, respectively.

Another aspect of this disclosure further relates to a pharmaceutical composition, comprising the above double-stranded RNA molecule.

In some embodiments, the pharmaceutical composition further comprises pharmaceutically acceptable carriers, diluents, and/or excipients.

The term “pharmaceutically acceptable” indicates that when administered to animals or humans properly, molecular bodies, molecular fragments, or compositions do not cause unfavorable, allergic, or other adverse effects. Specific examples of some substances that may be used as pharmaceutically acceptable carriers or components include phosphoric acid, citric acid, and other organic acids; antioxidants (for example, ascorbic acid and methionine); antimicrobial agents (for example, benzyldimethylstearylammonium chloride, hexamethonium chloride, benzalkonium chloride, phenol, butanol or benzyl alcohol, alkyl paraben, catechol, resorcinol, cyclohexanol, 3-pentanol, or m-cresol); peptides with low molecular weight (less than about 10 kDa); proteins such as serum albumin, gelatin, or immunoglobulins; hydrophilic polymers such as polyvinylpyrrolidone; amino acids (for example, glycine, glutamine, asparagine, histidine, arginine, or lysine); monosaccharides, disaccharides, and other carbohydrates (including, for example, glucose, mannose, or dextran); chelating agents (for example, EDTA); sugars (for example, sucrose, mannitol, trehalose, or sorbitol); salt counterion; metal composites; and/or nonionic surfactants (including, for example, TWEEN™, PLURONICS™, or polyethylene glycol). In addition, those of ordinary skills in the art can appropriately select common fillers, diluents, binders, humidifiers, disintegrants, and/or surfactants based on a preparation method. The pharmaceutical composition can be in a solid, semi-solid, or liquid form, and preferably, is in the liquid form.

In some embodiments, the pharmaceutical composition further comprises a nucleic acid stabilizer.

Examples of formulations for stabilizing and maintaining stabilization of nucleic acids include cationic compounds, detergents, chaotropic salts, ribonuclease inhibitors, chelating agents, and the like, and a mixture thereof. The stabilizers can include, for example, crosslinking fixatives such as paraformaldehyde or precipitants such as ethanol. The stabilizers can form covalent bonds between cell molecules, precipitate some intracellular molecules, or use other means to exert effects. In some embodiments, the stabilizers include cell lysis buffers. Cell permeabilization buffers are also known in the art and can include detergents that permeabilize cell membranes to allow probes and dyes to pass through the membranes. Examples of detergents used in the cell lysis buffers include, but are not limited to, TweeruTriton X-100, saponin, and NP-40. Concentrations of cell lysis buffers and permeabilizers are adjusted as per a given final purpose. When the cell lysis buffers and permeabilizers are at extremely low concentrations, optimal cell lysis and permeabilization may not be implemented. When the cell lysis buffers and permeabilizers are at extremely high concentrations, undesirable cell destruction may occur. Regular experiential steps can be performed to determine a preferred route in each case. In some embodiments, the stabilizers include chloroform, phenol, and TRIZOL. However, in a more preferred embodiment, the stabilizers are components that are easy to eliminate or that have lower cytotoxicity, and are most preferably pharmaceutically acceptable components.

In some embodiments, the pharmaceutical composition is packaged and delivered in a form of plasmids, viral vectors, liposomes, dendritic macromolecules, inorganic nanoparticles, or cell-penetrating peptides.

The double-stranded RNA molecule can be packaged directly, or can be packaged in its precursors. Plasmids and viral vectors may contain selective markers (for example, tags that facilitate enrichment, for example, his tags; or tags easy to detect, for example, GFP) and a replication origin matching a cell type specified for a cloning vector. In addition, an expression vector contains essential regulatory elements for affecting expression in the specified target cells. The viral vectors can be bacteriophage, lentivirus, retrovirus, adenovirus, or adeno-associated virus.

Liposomes can be cationic liposomes or neutral liposomes, which can be prepared or modified in a well-known method. For example, the addition of liposomes modified by polyethylene glycol (PEG) can effectively prevent aggregation of liposome carriers and increase their stability.

Dendritic macromolecules are a special family of polymers with definite molecular structures, precisely controllable chemical structures, and unique multivalent features, and therefore, are gradually becoming non-viral vectors for gene delivery. Typical dendritic macromolecules such as dendritic polymers of poly(amidoamine) (PAMAM) can be further modified. For example, a surface of PAMAM is modified with a nucleobase analogue 2-amino-6-chloropurine to construct a derivative AP-PAMAM. Alternatively, CS-PAMAM is prepared by conjugating chondroitin sulfate (CS) with PAMAM, etc.

Gold nanoparticles (AuNPs), magnetic nanoparticles, mesoporous silica nanoparticles (MSNs), and the like can be selected as inorganic nanoparticles.

Cell-penetrating peptides (CPPs) are a category of small molecule peptides with strong transmembrane transport abilities, and can transport various macromolecular substances such as polypeptides, proteins, and nucleic acids into cells. The cell-penetrating peptides can be cationic CPPs (for example, TAT, Penetratin, Polyarginine, P22N, DPV3, and DPV6), amphiphilic CPPs (which can be formed through covalent ligation of hydrophobic peptide sequences and NLSs or isolated from native proteins, for example, pVEC, ARF (1-22) and BPrPr (1-28)), and hydrophobic CPPs (usually containing only non-polar amino acid residues and having a net electric charge about 20% less than a total electric charge of the amino acid sequence).

The pharmaceutical composition may contain additional efficacious medicinal components, for example, therapeutic compounds used in the treatment of cancers, for example, fluorouracil. Technicians may select a proper pharmaceutically acceptable excipient based on a form of the pharmaceutical composition and know a method for preparing the pharmaceutical composition, and may select a proper method for preparing the pharmaceutical composition based on a type of the pharmaceutically acceptable excipient and a form of the pharmaceutical composition.

This disclosure further relates to use of the above double-stranded RNA molecule in preparation of a medicant for preventing or treating tumors.

In some embodiments, the medicant is used for inhibiting growth, proliferation, or metastasis of tumor cells.

The term “tumor” describes a physiological condition of a subject, where a cell population is characterized by unregulated (malignant or cancerous) cell growth. In some embodiments, the tumor is selected from ovarian cancer, rectal cancer, and liver cancer.

This disclosure further relates to a method for preventing or treating tumors, including a step of administering a safe efficacious dose of the above double-stranded RNA molecule or the pharmaceutical composition to a subject.

As used herein, the phrase “safe efficacious dose” means that a dose of a compound or a composition is greatly enough to alleviate a symptom or a condition significantly and effectively in treatment, but is small enough to avoid severe side effects (at a reasonable benefit-risk ratio) within a reasonable pharmaceutical regulation range. A safe efficacious dose of an active component in the pharmaceutical composition used in method in this disclosure varies along with factors including a specific treated symptom, an age and a physical condition of a treated subject, severity of a disease, duration of a treatment, a contemporaneous treatment, a specific used active component, a specific used pharmaceutically acceptable excipient, and knowledge and skills of the physician involved in the treatment.

As it is known to those skilled in the art, the double-stranded RNA molecule or the pharmaceutical composition in this disclosure can be administered via any route. In some embodiments, the pharmaceutical composition in this disclosure is administered via an oral (PO) route, an intravenous (IV) route, an intramuscular (IM) route, an intra-arterial route, an intramedullary route, an intrathecal route, a subcutaneous (SQ) route, an intraventricular route, a percutaneous route, an intradermal route, a transrectal (PR) route, a transvaginal route, an intraperitoneal (IP) route, an intragastric (IG) route, a topical route (through, for example, powders, ointments, creams, gels, lotions, and/or drops), a mucous membrane route, an intranasal route, an intrabuccal route, a transenteral route, a vitreous body, or a sublingual route; dripped via an endotracheal route, or dripped and/or inhaled via a bronchial route; or administered as an oral spray, a nasal spray, and/or an aerosol, and/or administered via a portal vein catheter. The double-stranded RNA molecule at a concentration of at least 3 nM, at least 5 nM, approximately 5 nM to approximately 200 nM, approximately 10 nM to approximately 100 nM, or approximately 25 nM to approximately 50 nM are provided in the composition.

The term “subject” in this disclosure may refer to a patient or another animal receiving an agent or the medicant in this disclosure for the treatment, prevention, alleviation and/or relief of the disease, the ailment, or the symptom in this application. The subject includes a warm-blooded animal, for example, a mammal such as a panda, an elephant, or a primate (a chimpanzee, an orangutan, a gibbon, a macaque, or a marmoset), and is, preferably, a human. Non-human primates are also individuals. The term “individuals” include domesticated animals such as cats and dogs, domestic animals (for example, cattle, horses, pigs, sheep, and goats), and experimental animals (for example, mice, rabbits, rats, gerbils, and guinea pigs).

The embodiments of this disclosure are described in detail below with reference to examples. It should be understood that these examples are only used to illustrate this disclosure and are not intended to limit the scope of this disclosure. In the following examples, for a test method for which a specific condition is not specified, preferably, refer to the guidance provided in this disclosure, a test manual or a conventional condition in the art, another test method known in the art, or a condition recommended by a manufacturer.

In the following specific examples, a measurement parameter related to an ingredient may have a slight deviation within a weighing accuracy range unless otherwise specified. For temperature and time parameters, an acceptable deviation due to instrument test accuracy or operation accuracy is allowed.

### Chemicals and Materials:

In the following examples, Ganoderma lucidum (Curtis: Fr.) P. Karst. were collected from the fresh mushrooms of Shaoguan City, Guangdong Province. Cetrimonium bromide (CTAB) and sodium chloride were purchased from Kingdin Industrial Co., Ltd. (Hong Kong, China). Water-saturated phenols were purchased from Leagene Co., Ltd. (Beijing, China). Chloroform and ethanol were purchased from Anaqua Chemicals Supply Inc. Ltd. (United States). Isoamyl alcohol and guanidine thiocyanate were purchased from Tokyo Chemical Industry CO., Ltd. (Japan). Tris-HCl and ethylenediaminetetraacetic acid (EDTA) were purchased from Acros Organics (United States). MicroRNA marker and low range ssRNA ladder were purchased from New England Biolabs (United States). Diethypyrocarbonate-treated RNase-free water, RNA T1 enzyme, 40% acrylamide/bis solution (19:1), tris/borate/EDTA (TBE), ammonium persulfate (APS), tetramethylethylenediamine (TEMED), mirVana™ miRNA isolation kit, SYBR Gold Nucleic Acid Gel Stain, and Gel Loading Buffer II were purchased from Thermo Fisher Scientific (United States). Guanidine thiocyanate, triethylamine, hexafluoroisopropanol, and fluorouracil were purchased from Sigma (United States). Ethanol was purchased from Anaqua Chemicals Supply Inc. Ltd. (United States). Deionized water was prepared via the Millipore Milli-Q Plus system (United States). The human ovarian cancer cell line (A2780) was purchased from KeyGen Biotech Co., Ltd. (Nanjing, China), and the human ileocecal colon adenocarcinoma cell line (HCT-8) and human hepatocellular carcinoma cell line (HepG2) were purchased from ATCC (United States). Opti-MEM low-serum medium, RPMI medium 1640, fetal bovine serum (FBS), and penicillin-streptomycin were purchased from Gibco (New Zealand). 3-(4,5-dimethylthiazol-2-yl)-2,5-diphenyltetrazolium bromide (MTT) was purchased from Sigma (St. Louis, MO, United States).

### Example 1 Isolation of RNA Molecules from Fruiting Bodies of Ganoderma lucidum (Curtis: Fr.) P. Karst

Fresh fruiting bodies of Ganoderma lucidum (Curtis: Fr.) P. Karst. were collected and stored immediately in liquid nitrogen before use. RNA with a length of 200 or less nucleotides, that is, small RNA, was extracted from Ganoderma lucidum (Curtis: Fr.) P. Karst. in an optimized Trizol method in combination with a commercial small RNA isolation kit. The method was described in Biomolecules (Yan, T., et al., 2020, 10, 621). In brief, fungal tissues were cut into small pieces, ground into a fine powder under liquid nitrogen, then homogenized in Trizol extraction buffer by using a digital dispersion apparatus (IKA, Germany) and allowed to stand and lyse for 10 minutes. Chloroform accounting for ⅓ of the volume was added, and a resulting mixture was shaken thoroughly, and allowed to stand for 10 minutes, and then centrifuged at 4000×g for 10 minutes. A supernatant was collected, 5M sodium chloride accounting for 1/25 of the volume and absolute ethanol accounting for 1.25 times the volume were added, a resulting mixture was allowed to stand at −20 degrees Celsius for 30 minutes, and centrifuged at 4000×g for 10 minutes, and a supernatant was discarded. A precipitate was washed with 80% ethanol and centrifuged at 4000×g for 10 minutes, and a supernatant was discarded.

After a lid was opened to evaporate ethanol, enzyme-free water was added and shaken until the precipitate was completely dissolved, an equal volume of CTAB was added, mixed well, and allowed to stand for 10 minutes, and then an equal volume of phenol: chloroform: isoamyl alcohol (50:48:1) was added for extraction via acute vortex oscillation. A resulting mixture was centrifuged at 4000×g for 15 minutes to separate the phases, and as described above, a supernatant was extracted again with chloroform: isoamyl alcohol (24:1). The supernatant was collected and mixed with an equal volume of 6M guanidine thiocyanate, and then 100% ethanol was added until a final concentration reached 55%. The mixture was filtered through a filter cartridge containing a silica membrane to fix RNA. The filter was then washed with 80% (v/v) ethanol solution several times, and finally, all RNAs were eluted with a low ionic strength solution or RNase-free water.

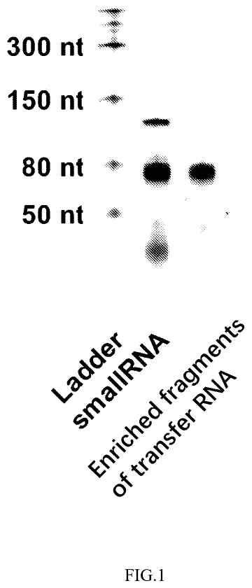

According to instructions of the manufacturer, the small RNAs were isolated and enriched by using the mirVana™ miRNA isolation kit. In addition, total tRNAs in isolated small RNAs were isolated via electrophoresis in a 6% polyacrylamide TBE gel containing 8M urea prepared based on a protocol of the manufacturer (Biorad, United States). After staining with SYBR Gold Nucleic Acid Gel Stain, polyacrylamide gel was inspected by using a UV lamp and a gel area containing the total tRNA was cut by a clean and sharp cutting plate. FIG. 1 shows a gel electrophoresis spectrum of a small RNA substance from Ganoderma lucidum (Curtis: Fr.) P. Karst., including a small RNA standard reference substance (denoted as a “Ladder”), a small RNA component, and enriched fragments of the transfer RNA.

A band was cut into small pieces and the total tRNA was recovered from the gel via electroelution in 1×TAE buffer at 100V for 90 minutes in a 3 kD molecular weight cut-off dialysis tube (Spectrum, C.A.). An eluate was recovered from the dialysis tube and the total tRNA was desalted and concentrated by using the mir Vana™ miRNA isolation kit. Quality and purity of the RNA product was then determined by using Nanodrop spectrophotometer (Thermo Scientific, United States) and Agilent 2100 bioanalyzer (Agilent, United States).

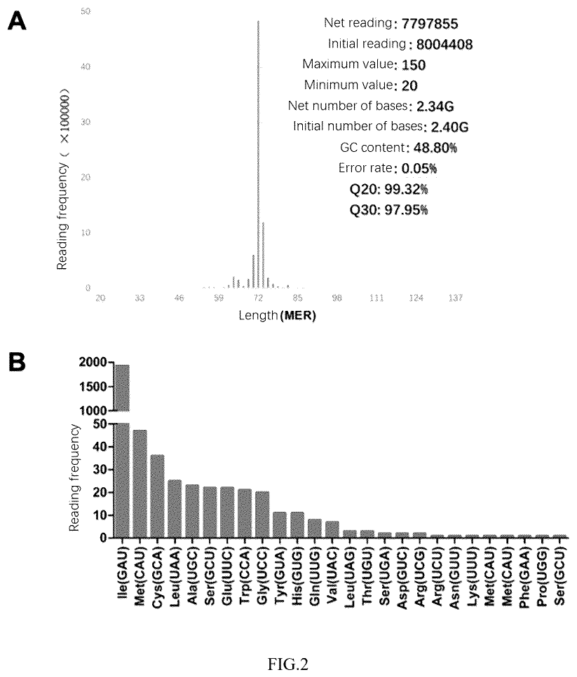

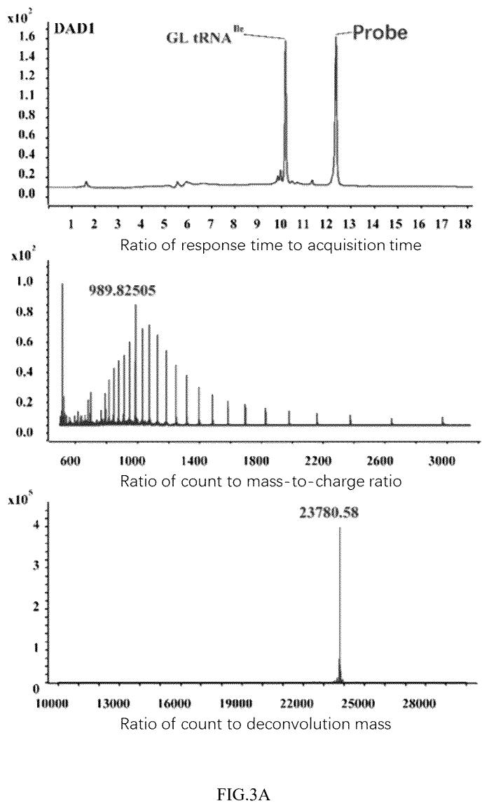

The inventor constructed and sequenced a total tRNA library. A sequencing library was generated via a round of adapter ligation, reverse transcription, and PCR enrichment by using TruSeq Small RNA Library Preparation Kit (Illumina, United States). The PCR product was then purified, and the library was quantified on Agilent Bioanalyzer 2100 system (Agilent Technologies, United States). The prepared library was sequenced at Novogene Bioinformatics Institute (Beijing, China) via a 150 bp pair-end (PE150) policy on Illumina HiSeq platform to generate over 8 million raw paired reads. A net reading of 7797855 was obtained by omitting low-quality areas and adapter sequences. FIG. A in FIG. 2 shows a bar chart of read length distribution of tRNAs. The tRNA genes were determined by using the tRNAscan-SE2.0 program (http://lowelab.ucsc.edu/tRNAscan-SE/) and annotated by searching a nucleotide collection (nr/nt) database via basic local alignment search tool (BLAST) program (https://blast.ncbi.nlm.nih.gov/Blast.cgi). 26 tRNA sequences derived from Ganoderma lucidum (Curtis: Fr.) P. Karst. were determined and are listed in Table 1, and reads of each type of tRNA are shown in FIG. B in FIG. 2.

Each type of tRNA was then isolated from a mixture of small RNAs (<200-mer) of Ganoderma lucidum (Curtis: Fr.) P. Karst. by fixing the target tRNA on streptavidin-coated magnetic beads via a specifically biotinylated capture DNA probe. To bind specific tRNA molecules, corresponding single-stranded DNA oligonucleotides (20- to 45-mer) were synthesized and designed based on sequence information in Illumina sequencing and should be complementary to a unique segment of the target tRNA. The homologous DNA probe and the small RNA mixture were incubated in an annealing buffer for approximately 1.5 hours and allowed to hybridize with the target tRNA molecule in a solution at proper annealing temperature, where the annealing temperature was usually 5° C. lower than the melting temperature (Tm). The streptavidin-coated magnetic beads were then added to the mixture and incubated at the annealing temperature for 30 minutes. After the hybridization sequence was fixed to the beads via streptavidin-biotin bonds, the biotinylated DNA/tRNA-coated beads were separated with a magnet for 1 or 2 minutes and washed 3 or 4 times in a wash buffer at 40° C. The beads were resuspended in RNase-free water until a desired concentration was reached, to release the fixed tRNA molecules via incubation at 70° C. for 5 minutes. As a result, various isolated and purified tRNA molecules were obtained.

### Example 2 Chemical Characterization of Purified RNA Molecules

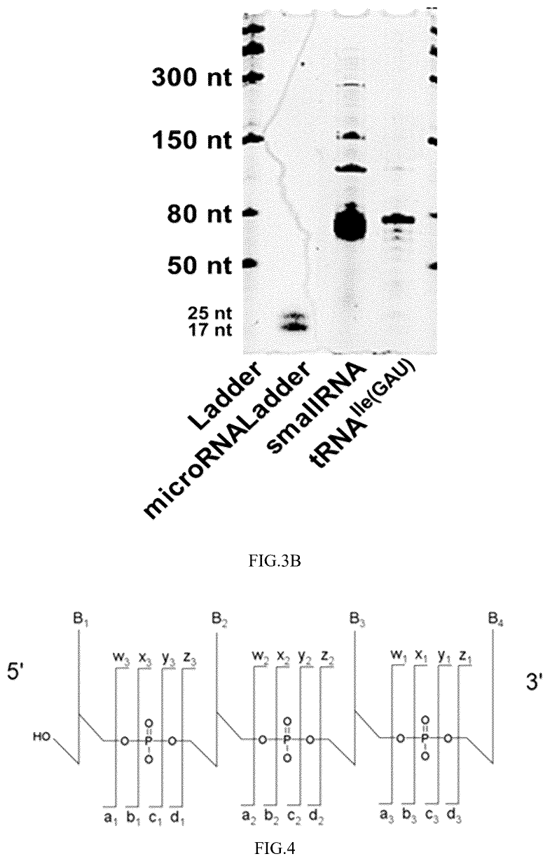

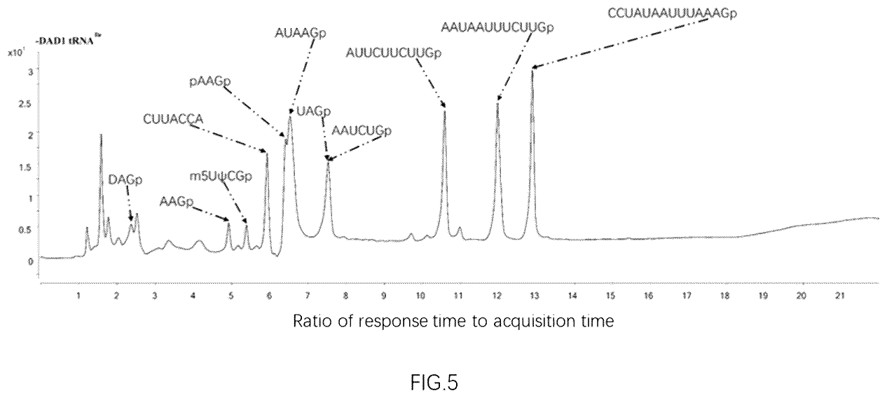

The inventor further qualitatively analyzed the sequence of the purified digested tRNA product and modification thereof by using a combined ultra-performance liquid chromatography-quadrupole-time-of-flight mass spectrometry (UHPLC-QTOF-MS) technology, to implement mass spectrometry characterization of tRNAs. After tRNAs were treated with endonuclease RNase T1, several oligonucleotide fragments that had 3′-phosphate of guanine nucleoside at their ends and had lengths of approximately 2 to 15 nt were generated. In the anion mode of the ESI source, the oligonucleotide generally produced excimer ion peaks with multiple electric charges, and the number of electric charges varies with a length of the oligonucleotide fragment. The longer the fragment, the greater the number of electric charges. Collision-induced dissociation (CID) was performed to analyze a mass spectrometry cleavage pattern and fragment information of fragments digested by tRNA-RNase T1 enzyme, to determine the oligonucleotide sequence and modification composition thereof. At the same collision voltage, the greater the intensity of the excimer ion peak, the greater the response intensity of the fragments. In addition, molecular ion peaks with more electric charges were more likely to be cleaved, and more sequence fragment information was generated. Because of complexity of CID spectra of oligonucleotide fragments with lengths greater than 8 nt, multiple excimer ions and corresponding optimal collision energy were selected for study in the sequence analysis process. CID cleavage of oligonucleotides was most likely to be implemented at phosphodiester bonds and ligation ends of bases and riboses, to generate a series of characteristic fragments, mainly including type-a-B, type-c, type-y, and type-w ions. As shown in FIG. 4, the sequences of oligonucleotides could be analyzed through type-c, type-y, and type-w ions, and types of nucleotide modifications could be further determined through type-a-B ions.

The prepared pure RNA was lyophilized and redissolved in RNase-free water treated with diethypyrocarbonate, to ensure that each 1 μg of pure RNA molecules were mixed with 50 units of RNase T1 enzyme, ammonium acetate was added until a concentration was 220 mM, a resulting mixture was incubated in a 37° C. water bath for 1.5 hours and then incubated at 70° C. for 10 minutes, and then the reaction was terminated. A resulting solution was centrifuged at 10000×g for 1 minute, and the supernatant was taken and subjected to the ultra-performance liquid chromatography-mass spectrometry analysis.

During the ultra-performance liquid chromatography-mass spectrometry analysis, Agilent UHPLC 1290 system (Agilent, United States) equipped with Agilent ultrahigh definition 6545 Q-TOF mass spectrometer was used. The chromatographic separation was based on ACQUITY UPLC OST C18 Column (with an inside diameter of 2.1 mm, a column length of 100 mm, and a padding material particle size of 1.7 μm, Waters, United States), mobile phases A were 100 mM hexafluoroisopropanol and 15 mM triethylamine, the mobile phase B was 50% methanol dissolved in the mobile phases A, a gradient elution procedure was performed from 0 to 1.5 minutes, the mobile phase B was maintained at 2% from 1.5 to 8.3 minutes, the mobile phase B was changed from 2% to 28% from 8.3 to 16.5 minutes, and the mobile phase B was changed from 28% to 34%. Ion source parameters: Air flow temperature was maintained at 320° C., the voltage was 3.5 kV, a sheath gas velocity was 12 L/min, and the sheath temperature was 350° C. Table 1 shows a secondary mass spectrometry result of fragments of digesting purified RNA molecules by specific RNA T1 enzyme, where a signal is consistent with that of a specific fragment of E. coli tRNA reported in the database. Table 4 shows identification of fragments of digesting purified RNA molecules by specific RNA T1 enzyme, where the purified RNA molecule is determined as tRNA^{Ile(GAU)} and a nucleotide sequence is denoted as SEQ ID NO: 1.

TABLE 1
Characteristic Fragments of tRNA^{Ile(GAU)} Digested by
T1 Enzyme and Secondary Mass Spectrometry Data

| | Characteristic | | Decon- | | |
|---|---|---|---|---|---|
| | fragments | Calculated | volution | | Measured |
| | digested by | massª | mass | | mass |
| tRNA | T1 enzyme | (Da) | (Da) | m/z | (Da) |
| Ile | PAAGp | 1101.622 | 1101.126 | [M-H]⁻ | 1100.1256 |
| (GAU) | | | | | |
| | CCUAUAAUUUAAAGp | 4479.690 | 4479.591 | [M-3H]³⁻ | 1492.1969 |
| | DAGp | 1000.621 | 1000.147 | [M-H]⁻ | 999.1466 |
| | AAUAAUUUCUUGp | 3822.257 | 3822.425 | [M-4H]⁴⁻ | 954.6062 |
| | AUAAGp | 1657.020 | 1657.223 | [M-2H]²⁻ | 827.6117 |
| | AAUCUGp | 1939.164 | 1939.239 | [M-2H]²⁻ | 968.6194 |
| | UAGp | 998.602 | 998.118 | [M-H]⁻ | 997.1176 |
| | AAGp | 1021.642 | 1021.161 | [M-H]⁻ | 1020.1610 |
| | TψCGp | 1294.773 | 1294.165 | [M-H]⁻ | 1293.1651 |
| | AUUCUUCUUGp | 3139.814 | 3139.352 | [M-3H]³⁻ | 1045.4508 |
| | CUUACCA | 2124.343 | 2124.323 | [M-2H]²⁻ | 1061.1615 |

### Example 3 Synthesis of Double-Stranded RNA Molecules

The inventor designed and synthesized a double-stranded tRNA-half (t-half) molecule with a length of 30 bp to 45 bp and a double-stranded RNA molecule with a length of 19 bp or 22 bp based on the sequence SEQ ID NO: 1 of Ile in the 26 sequences of tRNA of Ganoderma lucidum (Curtis: Fr.) P. Karst. in the database. As shown in Table 2, RNA molecules with antisense sequences selected from SEQ ID NOs: 2-10 and complementary sense sequences selected from SEQ ID NOs: 11-19 were designed and synthesized via cleavages at different sites on the tRNA sequences in Table 2. As shown in Table 3, RNA molecules with antisense sequences selected from SEQ ID NOs: 20-23 and complementary sense sequences selected from SEQ ID NOs: 24-27 were designed and synthesized via addition of chemical modifications at different nucleotide sites on the GL2 and GL6 sequences in Table 3.

TABLE 2
RNA Molecules That Are Derived from Sequences in
Table 1 and That Are Artificially Synthesized
Based on this Disclosure

| | SEQ | | SEQ | | |
|---|---|---|---|---|---|
| Coding | ID | Antisense sequence | ID | Sense sequence | |
| (mimic) | NO. | (5′ to 3′) | NO. | (5′ to 3′) | Length |
| GL1 | 2 | AAGCCUAUAAUUUAAAGG | 11 | CAAGAAAUUAUUCUA | 33 |
| | | UAGAAUAAUUUCUUG | | CCUUUAAAUUAUAGG | |
| | | | | CUU | |
| GL2 | 3 | AUAAGGAAUCUGUAGAAG | 12 | UGGUAAGCCCAAGAA | 41 |
| | | UUCGAUUCUUCUUGGGCU | | GAAUCGAACUUCUAC | |
| | | UACCA | | AGAUUCCUUAU | |
| GL3 | 4 | AUAAGGAAUCUGUAGAAG | 13 | UAAGCCCAAGAAGAA | 38 |
| | | UUCGAUUCUUCUUGGGCU | | UCGAACUUCUACAGA | |
| | | AU | | UUCCUUAU | |
| GL4 | 5 | AAGCCUAUAAUUUAAAGG | 14 | UCUACCUUUAAAUUA | 22 |
| | | UAGA | | UAGGCUU | |
| GL5 | 6 | AAGCCUAUAAUUUAAAGG | 15 | ACCUUUAAAUUAUAG | 19 |
| | | U | | GCUU | |
| GL6 | 7 | UCGAUUCUUCUUGGGCUU | 16 | UGGUAAGCCCAAGAA | 22 |
| | | ACCA | | GAAUCGA | |
| GL7 | 8 | AUUCUUCUUGGGCUUACC | 17 | UGGUAAGCCCAAGAA | 19 |
| | | A | | GAAU | |
| GL8 | 9 | AGUUCGAUUCUUCUUGGG | 18 | UAAGCCCAAGAAGAA | 22 |
| | | CUUA | | UCGAACU | |
| GL9 | 10 | UCGAUUCUUCUUGGGCUU | 19 | UAAGCCCAAGAAGAA | 19 |
| | | A | | UCGA | |

TABLE 3
RNA Molecules That Are Derived from (mimic) GL2 and
GL6 Sequences in Table 2 and That Are Artificially
Synthesized Based on this Disclosure

| Coding | SEQ ID | Antisense sequence | SEQ ID | Sense sequence | |
|---|---|---|---|---|---|
| (mimic) | NO. | (5′ to 3′) | NO. | (5′ to 3′) | Length |
| GL10 | 20 | AUAAGGAAUCUGUAGAA | 24 | UGGUAAGCCCAAGA | 41 |
| | | Gm5UwCGAUUCUUCUUG | | AGAAUCGAACUUCU | |
| | | GGCUUACCA | | ACAGAUUCCUUAU | |
| GL11 | 21 | AUAAGGAAUCUGUAGAA | 25 | UGGUAAGCCCAAGA | 41 |
| | | Gm5UUCGAUUCUUCUUG | | AGAAUCGAACUUCU | |
| | | GGCUUACCA | | ACAGAUUCCUUAU | |
| GL12 | 22 | AUAAGGAAUCUGUAGAA | 26 | UGGUAAGCCCAAGA | 41 |
| | | GUψCGAUUCUUCUUGGG | | AGAAUCGAACUUCU | |
| | | CUUACCA | | ACAGAUUCCUUAU | |
| GL13 | 23 | ψCGAUUCUUCUUGGGCU | 27 | UGGUAAGCCCAAGA | 22 |
| | | UACCA | | AGAAUCGA | |

ψ represents pseudouridine; and
m5U represents 5-methyluridine.

### Example 4 Cytotoxic Effects of tRNA-Half Mimic and tRF Mimic Molecules on Ovarian Cancer Cells, Colorectal Cancer Cells, and Liver Cancer Cells

A2780 and HCT-8 cell lines were cultured in RPMI 1640 medium containing 10% FBS and 1% penicillin/streptomycin. HepG2 cell lines were cultured in MEM medium containing 10% FBS and 1% penicillin/streptomycin. All the above cell lines were cultured at 37° C. in a humidified atmosphere containing 5% CO₂.

In the cytotoxicity analysis, exponentially growing cells of each cancer cell line were inoculated at a density of 5000 cells per well in a 100 μL medium in a 96-well microplate and allowed to adhere for 24 hours prior to treatment. Then obtained RNA molecules at successive concentrations with a nucleic acid transfer vector, namely, Lipofectamine™ RNAiMAX transfection reagent (Thermo Fisher Scientific, United States), were added to the cells. After 48 hours of treatment, MTT solution (50 μL per well, 1 mg/mL solution) was added to each well and incubated at 37° C. for 4 hours. Then 200 μL of dimethyl sulfoxide (DMSO) was added, and an optical density of the resulting solution was measured via calorimetry by using SpectraMax 190 microplate reader (Molecular Devices, United States) at 570 nm. Dose-response curves were obtained and IC₅₀ values were calculated via GraphPad Prism 5 (GraphPad, United States). Each experiment was performed three times.

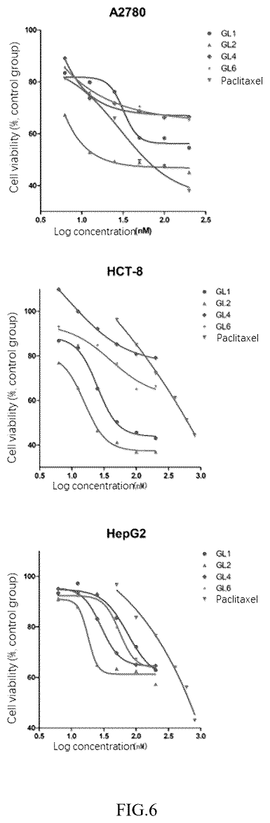

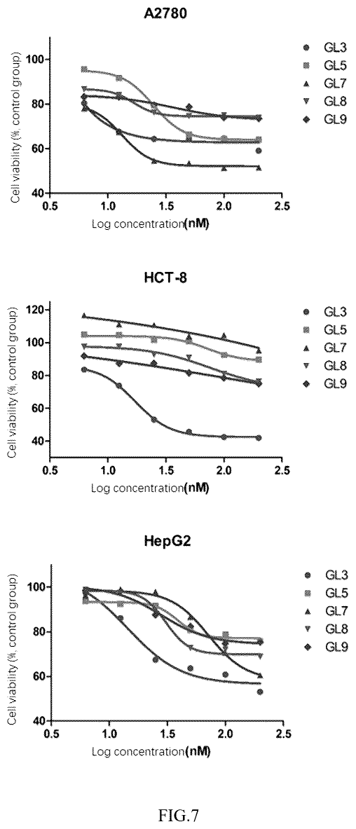

Before addition of the MTT solution, the inventor determined the cytotoxic effects and IC 50 of the GL1, GL2, GL4, and GL6 mimics at different concentrations of 6.25 nM, 12.5 nM, 25 nM, 50 nM, 100 nM, and 200 nM on A2780, HCT-8 and HepG2 cells. After 48 hours of the treatment of the double-stranded RNA molecules, the cell viability of these cells was compared with that of the control group and the RNAiMAX group. Paclitaxel was used in a comparative example. As shown in FIG. 6, these double-stranded RNA molecules are effective in inhibiting growth and proliferation of ovarian cancer cells, colon cancer cells, and liver cancer cells. GL3, GL5, GL7, GL8, and GL9 were tested in the same method, and results indicate that GL3, GL5, GL7, GL8, and GL9 also have the effect of inhibiting the growth and proliferation of cancer cells. Related results are shown in FIG. 7. The results were compared with those of the control group and the RNAiMAX group using the transfection agent. For IC₅₀, refer to Table 4. Paclitaxel was used in a comparative example.

TABLE 4
Median Inhibitory Concentrations IC₅₀ of GL1, GL2, GL4,
and GL6 Mimics for Ovarian Cancer Cells A2780, Colorectal
Cancer Cells HCT-8, and Liver Cancer Cells HepG2

| | IC₅₀ (nM) | | | | |
|---|---|---|---|---|---|
| Cell line | GL1 mimic | GL2 mimic | GL4 mimic | GL6 mimic | Taxol |
| A2780 | 225.0 | 57.23 | / | / | 73.25 |
| HCT-8 | 83.97 | 36.27 | 950 | 600 | 621.3 |
| HepG2 | 350 | 239.7 | 350 | 400 | 677.6 |

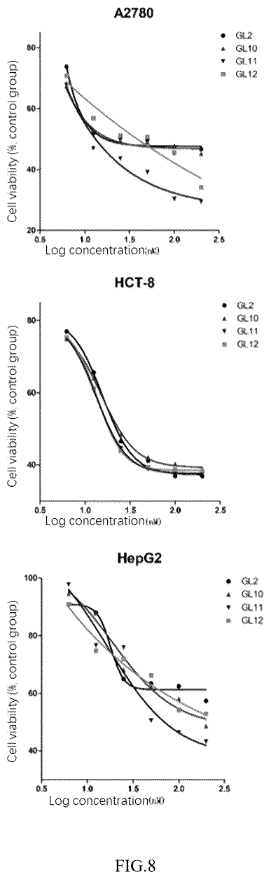

Then the inventor specifically determined the cytotoxic effects and IC₅₀ of the RNA molecules GL2 mimic and different chemically modified GL10, GL11 and GL12 mimics at different concentrations of 6.25 nM, 12.5 nM, 25 nM, 50 nM, 100 nM, and 200 nM on A2780, HCT-8 and HepG2 cells. As shown in FIG. 8, results were compared with those of the control group and the RNAiMAX group using the transfection agent. The results indicate that the RNA molecules GL2 mimic and different chemically modified GL10, GL11 and GL12 mimics have a dose-dependent effect on inhibiting the growth and proliferation of the ovarian cancer cells, the colon cancer cells, and the liver cancer cells. For IC₅₀, refer to Table 5. Paclitaxel was used in a comparative example.

TABLE 5
Median Inhibitory Concentrations IC₅₀ of GL2 Mimic and Different Chemically
Modified GL10, GL11, and GL12 Mimics for Ovarian Cancer Cells A2780,
Colorectal Cancer Cells HCT-8, and Liver Cancer Cells HepG2

| | IC₅₀ (nM) | | | | |
|---|---|---|---|---|---|
| Cell line | GL2 mimic | GL10 mimic | GL11 mimic | GL12 mimic | Taxol |
| A2780 | 57.23 | 51.93 | 17.18 | 43.73 | 73.25 |
| HCT-8 | 36.27 | 39.61 | 31.47 | 32.41 | 621.3 |
| HepG2 | 239.7 | 143.9 | 85.26 | 183.1 | 677.6 |

Only several examples of this disclosure are described in the above embodiments. The descriptions are relatively detailed and specific, but cannot be construed as a limitation on the patent scope of this disclosure. It should be noted that those of ordinary skills in the art could also make several variations and improvements without departing from the concept of the present disclosure. These variations and improvements all fall within the protection scope of the present disclosure. Therefore, the appended claims shall prevail over the protection scope of this disclosure, and the description and drawings can be used to illustrate content of the claims

1. A double-stranded RNA molecule, consisting of an antisense strand and a sense strand hybridized thereto, wherein a nucleotide sequence of the antisense strand is denoted as any one of SEQ ID NOs: 2-10.

2. The double-stranded RNA molecule according to claim 1, further comprising 3′ overhang.

3. The double-stranded RNA molecule according to claim 1, wherein the antisense strand and/or the sense strand of the double-stranded RNA molecule comprises one, two, or more modified nucleotides.

4. The double-stranded RNA molecule according to claim 3, wherein the modified nucleotide comprises one, two, or more of 4-acetylcytidine, 5-(carboxyhydroxymethyl)uridine, dihydrouridine, 2′-O-methylpseudouridine, β,D-galactose Q nucleoside, 2′-O-methylguanosine, inosinate, N6-isoprenyladenosine, 1-methyladenosine, 1-methylpseudouridine, 1-methylinosine, 2′2-dimethyl adenosine, 2-methyladenosine, 2-methylguanosine, 5-methyluridine, 3-methylcytidine, 5-methyl cytidine, N6-methyladenosine, 7-methylguanosine, 5-methylaminomethyluridine, 5-carboxymethyl aminomethyluridine, 5-carboxymethylaminomethyl-2-thiouridine, β,D-mannose Q nucleoside, 5-methoxycarbonylmethyl-2-thiouridine, 5-(methoxycarbonyl)methyluridine, 5-methoxyuridine, 2-thiomethyl-N6-isoprenyl adenosine, N-[(9-β-D-ribofuranosyl-2-methylthiopurin-6-yl) carbamoyl] threonine, N-[(9-β-D-ribofuranosylpurin-6-yl)N-methylcarbamoyl]threonine, uridine 5-oxyacetic acid methyl ester, uridine-5-oxyacetic acid, wybutoxosine, pseudouridine, Q nucleoside, 2-thiocytidine, 5-methyl-2-thiouridine, 2-thiouridine, 4-thiouridine, 5-thiouridine, N-[(9-β-D-ribofuranosyl-6-yl) carbamoyl]threonine, 2′-O-methyladenosine-5-methyluridine, 2′-O-methyl adenosine, 2′-O-methylcytidine, wybutosine, 3-(3-amino-3-carboxy-propyl)uridine, N6-acetyl adenosine, and 2-methylthio-N6-methyladenosine.

5. The double-stranded RNA molecule according to claim 3, wherein a nucleotide sequence of the antisense strand of the double-stranded RNA molecule is denoted as any one of SEQ ID NOs: 20-23, and a nucleotide sequence of a corresponding sense strand is denoted as any one of SEQ ID NOs: 24-27, respectively.

6. A pharmaceutical composition, comprising the double-stranded RNA molecule according to claim 1.

7. The pharmaceutical composition according to claim 6, further comprising a pharmaceutically acceptable carrier, diluent, and/or excipient.

8. The pharmaceutical composition according to claim 6, further comprising a nucleic acid stabilizer.

9. The pharmaceutical composition according to claim 6, wherein the pharmaceutical composition is packaged and delivered in a form of plasmids, viral vectors, liposomes, dendritic macromolecules, inorganic nanoparticles, or cell-penetrating peptides.

10. A method for preventing or treating tumors, comprising administrating a medicant comprising the double-stranded RNA molecule according to claim 1 to a subject in need thereof.

11. The method according to claim 10, wherein the medicant is used for inhibiting growth, proliferation, or metastasis of tumor cells.

12. The method according to claim 11, wherein the tumor is selected from ovarian cancer, rectal cancer, and liver cancer.
